(19) United States
(12) Patent Application Publication
(10) Pub. No.: US 2017/0258627 A1
(43) Pub. Date: Sep. 14, 2017

(54) ANTI-SNORING SYSTEMS

(71) Applicant: MARK CUZZETTO, BURNABY (CA)

(72) Inventor: MARK CUZZETTO, BURNABY (CA)

(21) Appl. No.: 15/068,859

(22) Filed: Mar. 14, 2016

(51) Int. Cl. A61F 5/56; A47C 31/00; A47G 9/02; A47G 9/10

(52) U.S. Cl. CPC A61F 5/56; A47G 9/1045; A47C 31/007; A47G 9/0253

(57) ABSTRACT

An anti-snoring system including an anti-snoring assembly. The anti-snoring assembly includes an anti-snoring-body; with the anti-snoring body including a front-side, a back-side, a top-side, and a bottom-side. The front-side includes a left-surface and a right-surface and the back-side includes a back-side-surface. The top-side includes a top-side-surface, where the bottom-side includes a bottom-side-surface. The anti-snoring system is structured and arranged to be placed beneath a pillow of a user to provide the user with a favorable downward-head-angle for reducing or preventing snoring during sleep. Embodiments include arrangements where the back-side is flat in shape, and both the top-side and the bottom-side are triangular in shape. Additionally, the left-surface and the right-surface of the front-side of the anti-snoring system may each resemble a flat-plane. Accordingly, the left-surface and the right-surface of the front-side of the anti-snoring system will be rectangular in shape.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of bedding materials and more specifically relates to anti-snoring systems.

2. Description of Related Art

Snoring is a condition which occurs during sleep and has been known to cause sleep deprivation to individuals that snore and those around them. Additionally, snoring may cause drowsiness, irritability, and lack of concentration during times of non-sleep. Sometimes snoring may be considered a minor affliction; however individuals who snore may sometimes suffer from a severe impairment of lifestyle. Recent medical studies have associated snoring with the development of carotid artery atherosclerosis.

Certain treatments and methods of reducing chances or instances of snoring can be found, which may include waking the snoring individual. However, waking a snoring individual may cause a sleep deprivation which may aggravate or increase instances of snoring. Some devices may be available to attempt to reduce chances of snoring; however, many of these devices are costly, complex and often ineffective. Therefore an inexpensive and simple to operate solution is desired.

Several attempts have been made to solve the above-mentioned problems such as those found in U.S. and Foreign Pat. and Pub. Nos. U.S. Pat. No. 4,848,360 to Palsgard et al., US 2014/0069777 to Derner, U.S. Pat. No. 7,469,434 to Drucker, U.S. Pat. No. 7,100,227 to Frisbee, U.S. Pat. No. 6,386,201 to Fard, U.S. Pat. No. 7,594,288 to Holliday et al., US 2012/0079660 to Chen, US 2012/0060846 to Leoniak et al., US 2014/0296747 to Herrnsdorf, US 2015/0265075 to Liu et al., CN 204,192,189 to Liang et al., CN 202,619,948 to Qi et al., CN 201,085,722 to Jianguo, CN 2,235,792 to Yong, EP 0765647 to Berends. DE 19,930,818 to Golriz, CA 2,478,289 to Koops, WO 01/32112 to Botha, and EP 1,488,766 to Screen. This art is representative of bedding accessories. However, none of the above inventions and patents, taken either singly or in combination, is seen to describe the invention as claimed.

Preferably, an anti-snoring system should provide device for providing a favorable head-position-angle during sleep to reduce or eliminate snoring and, yet would operate reliably and be manufactured at a modest expense. Thus, a need exists for a reliable anti-snoring system to avoid the above-mentioned problems.

### BRIEF SUMMARY OF THE INVENTION

In view of the foregoing disadvantages inherent in the known bedding materials art, the present invention provides a novel anti-snoring system. The general purpose of the present invention, which will be described subsequently in greater detail is to provide a device to be used in conjunction with a pillow to provide a user with a head position during sleeping that reduces or eliminates snoring.

An anti-snoring system is disclosed herein, in a preferred embodiment, the anti-snoring system comprising an anti-snoring assembly. The anti-snoring assembly may comprise an anti-snoring-body; with the anti-snoring body comprising a front-side, a back-side, a top-side, and a bottom-side in the preferred embodiment. The front-side may comprise a left-surface and a right-surface and the back-side may comprise a back-side-surface. The top-side may comprise a top-side-surface, where the bottom-side may comprise a bottom-side-surface. The anti-snoring system is structured and arranged to be placed beneath a pillow of a user useful for providing the user with a favorable downward-head-angle in order to reduce or prevent snoring during sleep.

Preferably, the back-side is flat in shape, and both the top-side and the bottom-side are substantially triangular in shape. Additionally the the left-surface and the right-surface of the front-side of the anti-snoring system may each comprise a flat-plane in the preferred embodiment. Accordingly, the left-surface and the right-surface of the front-side of the anti-snoring system may be rectangular in shape.

Preferred embodiments may include the anti-snoring system constructed of a washable material to provide the user with the ability to clean the anti-snoring system, and the anti-snoring system may be constructed of an anti-fungal material to improve hygiene. Also the anti-snoring system may be constructed of a flame-retardant material to provide increased safety to the user. Based on user preferences, embodiments may include the anti-snoring system constructed of non-organic closed-cell-foam material for increased longevity and comfort, or be constructed of hypo-allergenic materials to provide decreased allergens for the user.

A kit is also described herein. The kit includes: an anti-snoring system (comprising an anti-snoring assembly), a pillow, a pillow-case, and a set of user instructions.

A method of using an anti-snoring system is additionally disclosed herein, comprising the steps of providing an anti-snoring system, providing a pillow and pillow-case, removing the pillow from the pillow-case, placing the pillow on top of the anti-snoring system, inserting the anti-snoring system and the pillow into the pillow-case, placing the anti-snoring system, the pillow and the pillow-case upon a bed, and placing a head of a user upon the anti-snoring system, the pillow, and the pillow-case. The method may further include the optional steps of removing the head of the user from the anti-snoring system, the pillow and the pillow-case, and removing the anti-snoring system from the pillow-case.

The present invention holds significant improvements and serves as an anti-snoring system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, anti-snoring systems, constructed and operative according to the teachings of the present invention.

FIG. 1 shows a perspective view illustrating an anti-snoring system during an ‘in-use’ condition showing a user with the user's head placed upon an anti-snoring assembly according to an embodiment of the present invention.

FIG. 2 is a perspective view illustrating the anti-snoring system comprising an anti-snoring assembly according to an embodiment of the present invention of FIG. 1.

FIG. 3 is a perspective view illustrating a pillow placed upon the anti-snoring assembly according to an embodiment of the present invention of FIGS. 1-2.

FIG. 4 is a perspective view illustrating an embodiment of the anti-snoring assembly according to an embodiment of the present invention of FIGS. 1-3.

FIG. 5 is a flowchart illustrating a method of use for the anti-snoring system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a bedding materials and more particularly to an anti-snoring system as used to improve the prevention of instances of snoring during sleep for a user.

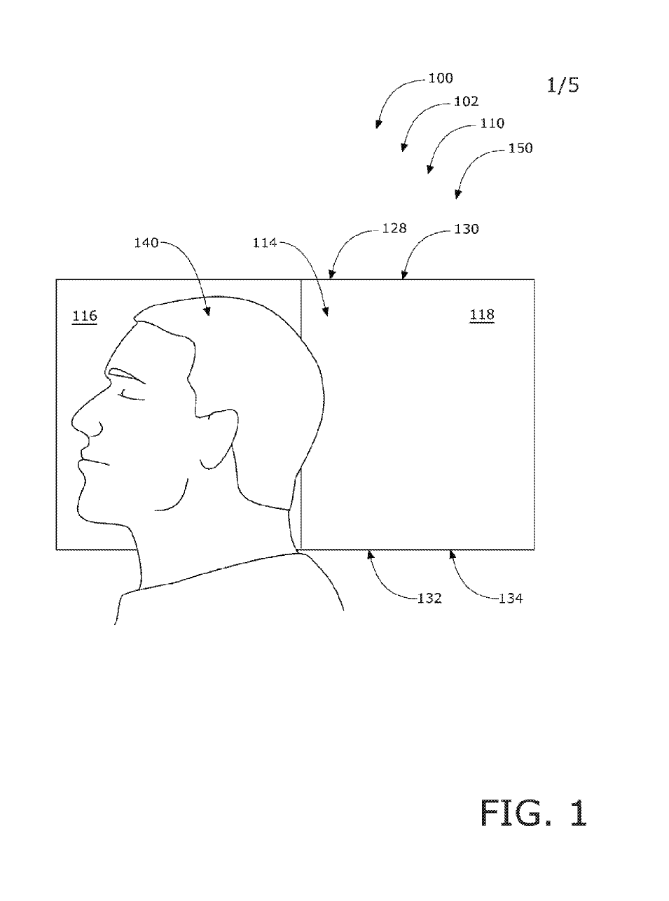

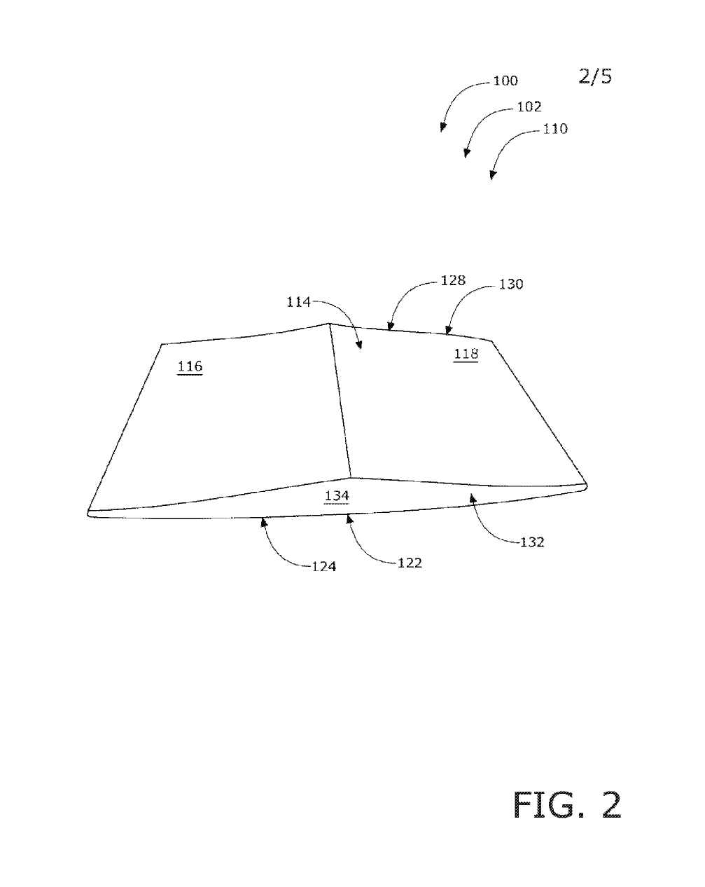

Referring to the drawings by numerals of reference there is shown in FIGS. 1-4, anti-snoring system 100 comprising anti-snoring assembly 102. As shown in FIG. 2, anti-snoring assembly 102 may comprise anti-snoring-body 110, with anti-snoring body 110 comprising and defined by front-side 114, back-side 122, top-side 128, and bottom-side 132. Front-side 114 may comprise and be defined by left-surface 116 and right-surface 118. Back-side 122 may comprise and be defined by back-side-surface 124. Top-side 128 may comprise and be defined top-side-surface 130 and, similarly, bottom-side 132 may comprise and be defined by bottom-side-surface 134.

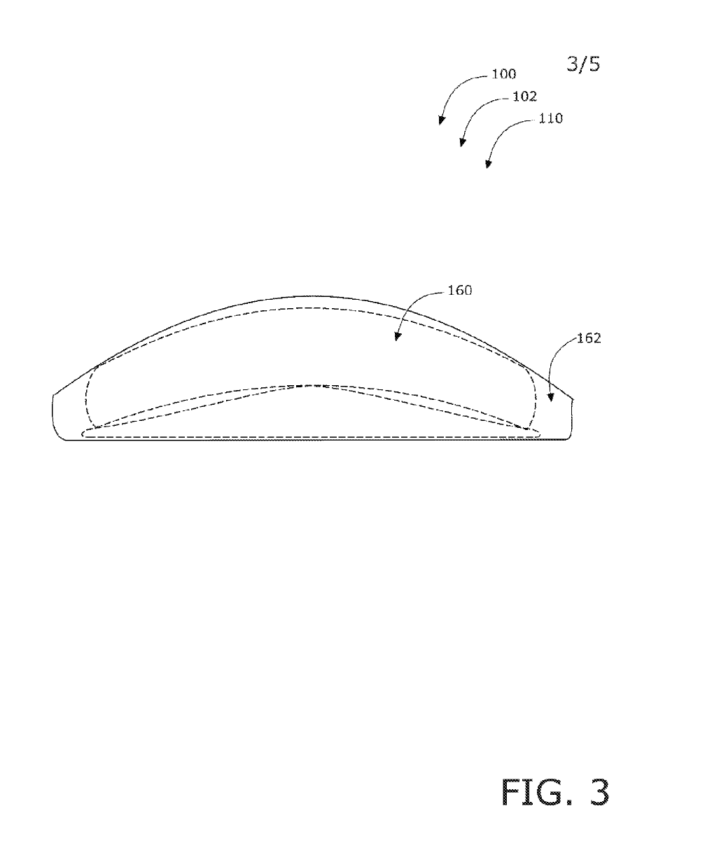

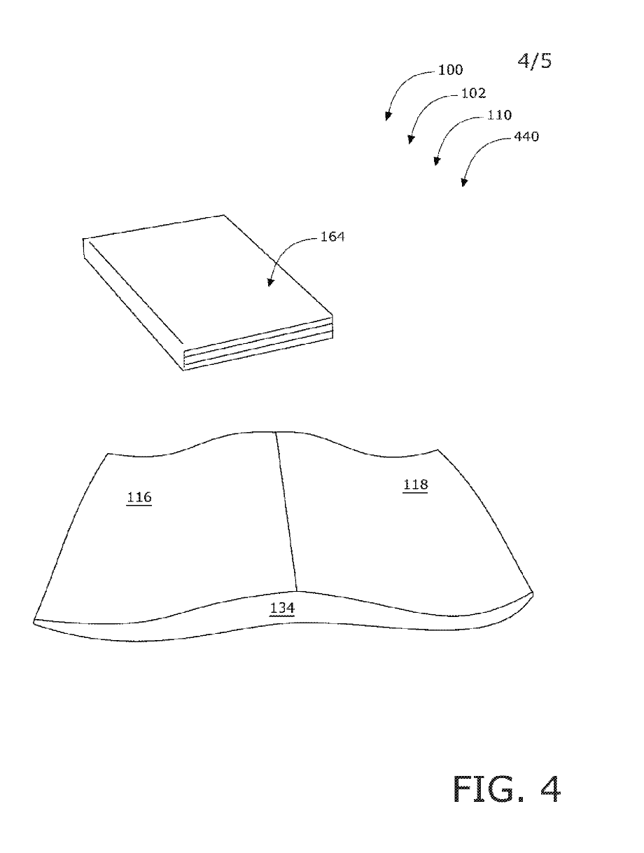

As shown in FIG. 3, anti-snoring system 100 is structured and arranged to be placed beneath pillow 160 of user during an ‘in-use’ 150 condition, useful for providing user with a favorable downward-head-angle, the favorable-downward head angle useful for reducing and preventing snoring during sleep. Embodiments may include back-side 122, which may be planar in shape, and top-side 128 and bottom-side 132, where each may be triangular in shape, as can be seen in FIG. 3. In such embodiments, left-surface 116 and right-surface 118 of front-side 114 of anti-snoring system 100 each comprise a flat-plane. Alternately, left-surface 116 and right-surface 118 of front-side 114 of anti-snoring system 100 may each comprise a curvilinear plane (as can be seen in FIG. 4).

Left-surface 116 and right-surface 118 of front-side 114 of anti-snoring system 100 may be square in shape, or left-surface 116 and right-surface 118 of front-side 114 of anti-snoring system 100 may be rectangular in shape. Also, left-surface 116 and right-surface 118 of front-side 114 of anti-snoring system may be triangular in shape, or left-surface 116 and right-surface 118 of front-side 114 of anti-snoring system 100 may be semi-circular in shape, depending upon user preferences and manufacturing methods.

Most embodiments of anti-snoring system 100 are of a size similar to that of a standard pillow 160 used for sleeping. Preferably, anti-snoring system 100 is constructed of a washable material to provide user with the ability to clean anti-snoring assembly 102 and may additionally be constructed of an anti-fungal material to improve hygiene and/or constructed of a material which is flame-retardant to increase safety.

Materials of manufacture of anti-snoring system 100 may vary, such that anti-snoring system 100 may be constructed of a non-organic, closed-cell-foam material for increased longevity and comfort to user or may be constructed of an organic closed-cell-foam material to provide increase desirability for specific users. Additional materials of anti-snoring system 100 may include hypo-allergenic materials to provide decreased allergens for user. Other materials may be used and different shapes/profiles may apply.

Anti-snoring system 100 may be sold as a kit 440 comprising the following parts: at least one anti-snoring assembly 102, at least one pillow 160; at least one pillow-case 162; and at least one set of user instructions 164. The kit has instructions such that functional relationships are detailed in relation to the structure of the invention (such that the invention can be used, maintained, or the like in a preferred manner). Anti-snoring 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different pillow combinations, parts may be sold separately, etc., may be sufficient.

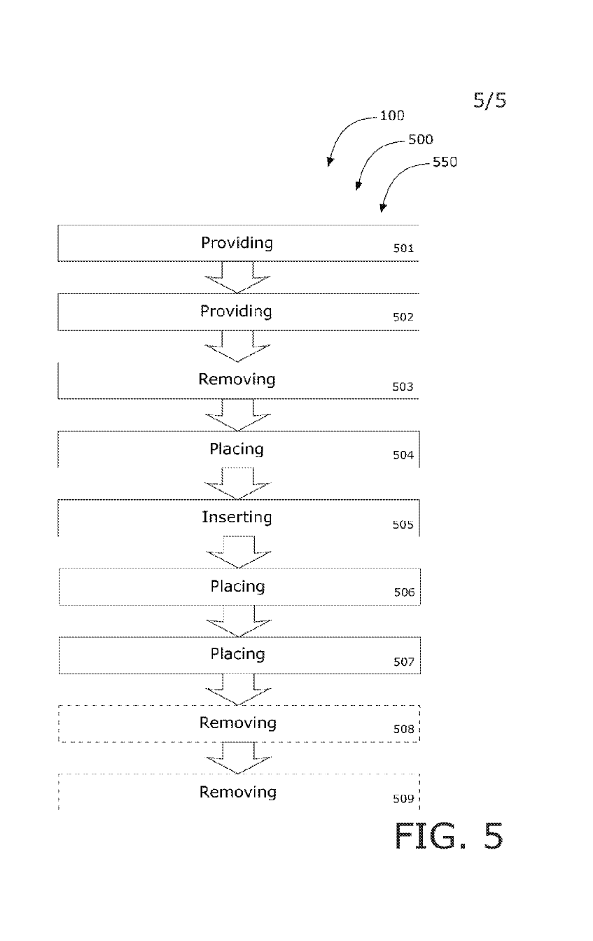

Referring now to FIG. 5 showing flowchart 550 illustrating method of use 500 for anti-snoring system 100 according to an embodiment of the present invention of FIGS. 1-4.

As shown, method of use 500 may comprise the steps of: step one 501, providing anti-snoring assembly 102; step two 502, providing pillow 160 and pillow-case 162; step three 503, removing pillow 160 from pillow-case 162; step four 504, placing pillow 160 on top of anti-snoring assembly 102; step five 505, inserting anti-snoring assembly 102 and pillow 160 into pillow-case 162; step six 506, placing anti-snoring assembly 102, pillow 160 and pillow-case 162 upon a bed of user 140; step seven 507, placing head of user 140 upon said anti-snoring assembly 102, pillow 160 and pillow-case 162; step eight 508, removing the head of user 140from anti-snoring assembly 102, pillow 160 and pillow-case 162; and step nine 509, removing anti-snoring assembly from pillow-case 162.

It should be noted that step eight 508 and step nine 509 are each optional steps and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶ 6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An anti-snoring system comprising:
an anti-snoring assembly comprising;
an anti-snoring-body comprising;
a front-side comprising;
a left-surface; and
a right-surface;
a back-side comprising;
a back-side-surface;
a top-side comprising;
a top-side-surface; and
a bottom-side comprising;
a bottom-side-surface;
wherein said anti-snoring system comprises said anti-snoring assembly;
wherein said anti-snoring assembly comprises said anti-snoring-body;
wherein said anti-snoring-body is defined by said front-side, said back-side, said top-side, and said bottom-side;
wherein said front-side is defined by said left-surface and said right-surface;
wherein said back-side comprises said back-side-surface;
wherein said top-side comprises said top-side-surface;
wherein said bottom-side comprises said bottom-side-surface; and
wherein said anti-snoring system is structured and arranged to be placed beneath a pillow of a user useful for providing said user with a favorable downward-head-angle useful for reducing and preventing snoring during sleep.

2. The anti-snoring system of claim 1 wherein said back-side is planar in shape.

3. The anti-snoring system of claim 2 wherein said top-side and said bottom-side are triangular in shape.

4. The anti-snoring system of claim 3 wherein said left-surface and said right-surface of said front-side of said anti-snoring system each comprise a flat-plane.

5. The anti-snoring system of claim 2 wherein said left-surface and said right-surface of said front-side of said anti-snoring system each comprise a curvilinear plane.

6. The anti-snoring system of claim 2 wherein said left-surface and said right-surface of said front-side of said anti-snoring system are square in shape.

7. The anti-snoring system of claim 3 wherein said left-surface and said right-surface of said front-side of said anti-snoring system are rectangular in shape.

8. The anti-snoring system of claim 1 wherein said left-surface and said right-surface of said front-side of said anti-snoring system are triangular in shape.

9. The anti-snoring system of claim 1 wherein said left-surface and said right-surface of said front-side of said anti-snoring system are semi-circular in shape.

10. The anti-snoring system of claim 1 wherein said anti-snoring system is of a size similar to that of a standard pillow used for sleeping.

11. The anti-snoring system of claim 1 wherein said anti-snoring system is constructed of a washable material to provide said user with the ability to clean said anti-snoring assembly.

12. The anti-snoring system of claim 1 wherein said anti-snoring system is constructed of an anti-fungal material.

13. The anti-snoring system of claim 1 wherein said anti-snoring system is constructed of non-organic closed-cell-foam material for increased longevity and comfort to said user.

14. The anti-snoring system of claim 1 wherein said anti-snoring system is constructed from an organic closed-cell-foam material to provide increase desirability for specific said users.

15. The anti-snoring system of claim 1 wherein said anti-snoring system is constructed of a flame-retardant material to provide increased safety to said user.

16. The anti-snoring system of claim 1 wherein said anti-snoring system is constructed of hypo-allergenic materials to provide decreased allergens for said user.

17. An anti-snoring system comprising:
an anti-snoring assembly comprising;
an anti-snoring-body comprising;
a front-side comprising;
a left-surface; and
a right-surface;
a back-side comprising;
a back-side-surface;
a top-side comprising;
a top-side-surface;
a bottom-side comprising; and
a bottom-side-surface;
wherein said anti-snoring system comprises said anti-snoring assembly;
wherein said anti-snoring assembly comprises said anti-snoring-body;
wherein said anti-snoring-body is defined by said front-side, said back-side, said top-side, and said bottom-side;
wherein said front-side is defined by said left-surface and said right-surface;
wherein said back-side comprises said back-side-surface;
wherein said top-side comprises said top-side-surface;
wherein said bottom-side comprises said bottom-side-surface;
wherein said anti-snoring system is structured and arranged to be placed beneath a pillow of a user useful for providing said user with a favorable downward-head-angle useful for reducing or preventing snoring during sleep;
wherein said back-side is flat in shape;
wherein said top-side and said bottom-side are triangular in shape;
wherein said left-surface and said right-surface of said front-side of said anti-snoring system each comprise a flat-plane;
wherein said left-surface and said right-surface of said front-side of said anti-snoring system are rectangular in shape;
wherein said anti-snoring system is constructed of a washable material to provide said user with the ability to clean said anti-snoring system;
wherein said anti-snoring system is constructed of an anti-fungal material;
wherein said anti-snoring system is constructed of non-organic closed-cell-foam material for increased longevity and comfort to said user;
wherein said anti-snoring system is constructed of a flame-retardant material to provide increased safety to said user; and
wherein said anti-snoring system is constructed of hypo-allergenic materials to provide decreased allergens for said user.

18. The anti-snoring system of claim 17 further comprising a kit including:
said anti-snoring assembly;
a pillow;
a pillow-case; and
a set of user instructions.

19. A method of using an anti-snoring system comprising the steps of:
providing an anti-snoring assembly;
providing a pillow and pillow-case;
removing said pillow from said pillow-case;
placing said pillow on top of said anti-snoring assembly;
inserting said anti-snoring assembly and said pillow into said pillow-case;
placing said anti-snoring assembly, said pillow and said pillow-case upon a bed of said user; and
placing a head of a user upon said anti-snoring assembly, said pillow and said pillow-case.

20. The method of claim 19 further comprising the steps of:
removing said head of said user from said anti-snoring assembly, said pillow and said pillow-case; and
removing said anti-snoring assembly from said pillow-case.
